(19) United States
(12) Patent Application Publication
(10) Pub. No.: US 2017/0028118 A1
(43) Pub. Date: Feb. 2, 2017

(54) A PROCESS FOR THE ESTIMATION OF TREATMENT DURATION OF PERITONEAL DIALYSIS TREATMENT

(71) Applicant: FRESENIUS MEDICAL CARE DEUTSCHLAND GMBH, Bad Homburg (DE)

(72) Inventors: Erik GRIESSMANN, Schweinfurt (DE); Martin BUDA, Dittelbrunn (DE); Klaus WOLF, Arnstein (DE)

(73) Assignee: FRESENIUS MEDICAL CARE DEUTSCHLAND GMBH, Bad Homburg (DE)

(21) Appl. No.: 15/302,617

(22) PCT Filed: Mar. 31, 2015
(86) PCT No.: PCT/EP2015/000698

§ 371 Date: Oct. 7, 2016

(30) Foreign Application Priority Data
Apr. 8, 2014 (DE) 10 2014 005 122.6

(51) Int. Cl. A61M 1/28

(52) U.S. Cl. CPC A61M 1/282; A61M 1/285; A61M 2205/3334; A61M 2205/3331; A61M 2205/50; A61M 2205/52

(57) ABSTRACT

The present invention relates to a process for the estimation of treatment duration of peritoneal dialysis treatment: wherein the estimation of treatment duration is performed based on an ideal treatment duration, wherein the ideal treatment duration is increased by one or more delay values, wherein the delay values depend on one or more apparatus-specific and/or patient-specific and/or process-specific parameters.

### CROSS-REFERENCE TO RELATED APPLICATIONS

This application is a National Stage of PCT application PCT/EP2015/000698, filed on Mar. 31, 2015, which claims priority to German Application No. 10 2014 005 122, filed on Apr. 8, 2015, the entirety of which is hereby incorporated by reference.

### FIELD OF THE INVENTION

The present invention relates to a process for the estimation of treatment duration of peritoneal dialysis treatment.

In the context of a peritoneal dialysis treatment, a dialysis solution is introduced into the patient's abdominal cavity for the removal of harmful substances from the blood. For this, an operably inserted catheter is employed by means of which the sterile dialysis solution is introduced. This dialysis solution receives harmful substances through the peritoneum acting as a semi-permeable membrane, i.e. through the patient's peritoneum, from the patient's blood. In addition, the patient is deprived of water due to glucose contained in the dialysis solution and the osmotic pressure resulting therefrom. After a certain dwell time in the abdominal cavity, the solution is withdrawn again through the catheter and replaced by a fresh dialysis solution.

Different methods of peritoneal dialysis are known from prior art, such as for example continuous ambulatory peritoneal dialysis (CAPD), wherein patients with the help of a bag system replace the dialysis solution by themselves about 4 to 5 times a day. In automated peritoneal dialysis this is performed by a dialysis apparatus, called cycler, so that even overnight treatment is enabled.

### SUMMARY OF THE INVENTION

In order to have better planning security for both nurses and patients (transportation services, machine times etc.) it would be desirable to have a reliable method enabling estimation of the overall treatment duration of a peritoneal dialysis treatment. It is thus an object of the present invention to provide a process for the estimation of the prospective treatment duration of a peritoneal dialysis treatment.

This object will be achieved by a process having the characteristics of claim 1.

Thus, provision is made for the estimation of treatment duration to be performed based on an ideal treatment duration, wherein the ideal treatment duration is increased by one or more delay times depending on one or more parameters, which are specific to a patient and/or a process and/or an apparatus.

These delay times may be absolute time spans or may be relative time spans, such as for example percentage of increase of introduction period or outflow period.

Thus, according to the invention, an ideal treatment duration will initially be determined. If, by way of example, the peritoneal dialysis treatment is considered to include an initial outflow, two cycles each having an inflow, a dwell time in the abdominal cavity, an outflow and a final inflow, then the ideal treatment duration is determined by adding up the duration of the initial outflow, the duration of one cycle multiplied by the number of cycles performed, as well as the duration of the final inflow. Based on this time, according to the invention, surcharges, i.e. factors entailing increased treatment duration, are taken into account as delay times, in order to achieve estimation of the overall treatment duration.

The magnitude of time variation between the actually performed treatment and the prescription normally depends on the characteristics of the patient and the characteristics of the catheter, respectively. It is thus conceivable that the expected flow rate will not be achieved. Another variation results from the fact that the patient generates an ultrafiltration volume so that the expected outflow volume increases.

Since there is no preset outflow volume for pressure controlled outflows the volume present in the patient will be assumed for the determination of the outflow volume.

In a preferred embodiment of the invention provision is made for the ideal treatment duration to comprise the initial duration of the outflow, the ideal cycle duration multiplied by the number of cycles as well as the ideal duration of the last inflow. With a pressure-controlled outflow the ideal duration of the initial outflow may be assumed to have a specific value of e.g. 5 min. With a pressure-controlled outflow no specific outflow volume is preset but the patient is emptied as long as any volume may be discharged from the abdominal cavity.

The ideal cycle duration preferably is composed of the ideal inflow duration, ideal dwell time as well as the ideal outflow duration. The ideal inflow duration may be determined as a quotient from the inflow volume and the inflow rate. The dwell time may be preset. The ideal outflow duration may be determined from the quotient of the set outflow volume and the outflow rate.

To account for the time-dependent operational state of the catheter, in a preferred embodiment of the invention, forming a patient-specific parameter by the catheter performance is provided, which may cause delay in relation to the ideal treatment duration. Even if the catheter performance during inflow depends on the individual patient to a lesser extent it nevertheless is considered to be one of the patient-specific parameters as the catheter broadly is a component of the patient.

Preferably, provision is made for the catheter performance to be taken into account for the estimation of inflow duration and/or outflow duration and/or duration of the last inflow.

It is to be considered that the catheter performance is taken into account in the form of absolute or relative increase in ideal inflow duration and/or ideal outflow duration and/or ideal duration of the last inflow. If for example one assumes that the ideal inflow duration is 4 min (inflow volume: 1000 ml; inflow rate: 250 ml/min), catheter performance may for example be taken into account by the factor of 110% so that as an estimated inflow duration a time of 264 s will result.

A corresponding approach is to be taken into account for the outflow duration as well as duration of the last inflow. If for the duration of initial outflow no fixed time span is assumed, i.e. the initial outflow is performed by volume control, increase of the ideal time span is as well to be taken into account accordingly.

Catheter performance and especially absolute or relative increase may be editable so that a user may be able to set these values. It is therefore conceivable to assume different values for different phases. Thus, for example relative delay in inflow may be 10%, and, on the other hand, may be 50% in outflow. It is to be considered to specify default values, which subsequently may be varied by the user if necessary.

The inflow delay due to not having reached any ideal inflow rate is patient-dependent to a lesser extent, so that provision may be made for it not to be editable by the apparatus, i.e. not to be changeable by a user, but to be fixedly set due to experience values, such as e.g. to be set to a value of 10% stated above.

It is an object of the invention that only few parameters relevant to the delay may be user-changeable or that all or none of these parameters may be user-changeable. In the latter case, delay values may all be fixedly set on the apparatus.

As stated above, provision may be made for the initial outflow to be performed in a pressure-controlled manner and the duration of the initial outflow to be preset by the apparatus or to be able to be set by a user. Thus, for example a value of 5 min is specified for the initial outflow.

The invention furthermore relates to the case, wherein the initial outflow is performed in a volume controlled manner and the duration of the initial outflow is determined from an (estimated) initial outflow volume and outflow rate. The duration of the initial outflow may as well be increased by an absolute or relative delay value to account for the catheter performance.

The process-specific parameter may for example be an absolute or relative delay during inflow and/or it may be a delay during outflow. Even these values may fixedly be preset or may be changeable by any user. It may for example be taken into account that a delay of 1 min per outflow and a delay of 2 min per inflow may be set. Consequently, these process-related delays thus do not depend on the volume, which has been passed through.

If for example as an ideal inflow duration a time span of 4 min is assumed then the catheter performance may be increased e.g. by an additional 10%. If one assumes a process-related delay of e.g. 120 s then an inflow duration of 384 s (=240 s*110%+120 s) will result.

One unknown is represented by the volume for the initial outflow if this has been prescribed as a pressure-controlled outflow. During this, the patient is emptied until there is no more volume to be passed through the catheter into the drainage. If, based on this assumption and having knowledge of the feed rate, a specific duration is assumed for the initial outflow, such as e.g. 5 min, then a theoretical volume may be determined, which may be passed through during this specified period of time. The volume of the initial outflow may thus be determined from the effective duration of the initial outflow and from the effective outflow rate.

The duration of the initial outflow may thereby be determined from a preset time including any procedural delay, e.g. of the amount of 1 min.

It may furthermore be taken into account that the effective outflow rate is determined from the preset outflow rate and a value accounting for the catheter performance, which e.g. is determined in the form of percentage surcharge.

The volume of the initial outflow determined in this manner may be used to establish a graph showing the volume of dialysis liquid present in the patient over the time.

Furthermore, it may be taken into account that for the estimated duration of dwell time of the solution in the patient and/or for the estimated duration of a treatment break the preset duration is employed. If for example for the dwell time a value of 60 min is preset, this value may as well be used for the estimation, since extension of dwell time is not expected to occur. Similarly, the same is true for treatment breaks.

For the estimated duration of a message the value of zero may be assumed since a message is not taken into account for the treatment duration.

According to a preferred embodiment of the invention the estimated treatment duration includes the following estimated periods of time, wherein the indicated durations or phases, respectively, may be performed not at all, exactly one time or several times, depending on the course of treatment: initial outflow duration, inflow duration, outflow duration, in patient dwell time, final outflow duration, duration of treatment break, duration of messages, wherein for the latter preferably the value of zero is assumed and wherein for the in-patient dwell time and for the break of treatment duration preferably the preset, i.e. the known values are used.

In any further embodiment of the invention provision is made for effective inflow rates and/or outflow rates to be determined, and for the purpose of establishing any tendency analysis by documentation of these data over time a change in catheter performance is determined. If the volume of the treatment liquid in the patient is plotted over the time then the slope of the graph represents the inflow rate or the outflow rate, respectively. If documentation over an extended period of time is performed, one may predict, whether the rates and consequently the catheter performance have changed.

In order to be able give evaluated values as realistic as possible, provision may be made for the treatment duration for future treatments to be based on the catheter performance, which has been changed. Thus, it may for example be taken into account, for the outflow delay, instead of setting a factor of 150%, to rather set 160%, if it has been shown, that the catheter performance has decreased accordingly.

It may furthermore be taken into account that the estimated treatment duration will be compared to an upper and/or lower threshold value and that information will be edited to the user if the estimated treatment duration exceeds the upper threshold value or falls below the lower threshold value. In this way, it may be determined if the envisaged treatment is to be performed under realistic specifications or if correction of one or more parameters is required.

In a further embodiment of the invention provision is made for the dwell time of the treatment to be changed or to remain unchanged during the estimated treatment duration. Thus, in the course of performing treatments dwell times, i.e. the time spans, during which the dialysis liquid is present in the abdominal cavity, may occasionally be reduced. If for example as a total treatment duration a specific period of time is set and if the estimated treatment duration exceeds this set period of time, provision may be made for the dwell time(s) to accordingly become reduced such that the preset period of time is met, i.e. the estimation using corrected dwell times exactly or largely corresponds to the preset period of time, which also includes that the estimation is in an allowable range.

If on the other hand, the estimated duration is in an allowable range or if it corresponds to an allowable value, then modification of the dwell times is not required and treatment may be performed as envisaged.

The present invention furthermore relates to a dialysis apparatus for performing peritoneal dialysis, having at least one logic or control unit, at least one input unit for the input of one or more values, as well as at least one processor, wherein the processor is programmed to perform a process according to one of the claims 1 to 14. Furthermore the dialysis apparatus preferably has one or more memories for reliably saving e.g. any threshold values of allowable ranges or for saving preset values, e.g. any value preset by the apparatus for a delay e.g. in the course of inflow etc. The memory may also serve for saving user-entered data.

In a preferred embodiment of the invention provision is made for the dialysis apparatus to have at least one device formed such that it determines a course of time of the dialysis solution volume in the patient. This course of time may subsequently be printed or may visibly be displayed to any user.

It may also be taken into account, that the apparatus has means, which are formed such that they determine the slope of the volume increase (during inflow) and/or of the volume decrease (during outflow) and which compares several temporally spaced apart slopes to each other. From this comparison it may subsequently be deduced, if any change of the slope has occurred resulting from a change in catheter performance. If this is the case, a change of default values may be performed by the apparatus, or the user may be provided with a suggestion for a value for the catheter performance.

The statements concerning the dialysis apparatus are also true for the process according to the invention.

### DESCRIPTION OF THE DRAWINGS

Further details and advantages of the invention will be explained in detail by way of an example shown in FIG. 1, wherein:

FIG. 1 shows a time course of the volume of a dialysis solution according to an estimation applied to a patient as well as the actual time course of the dialysis solution volume applied to the patient.

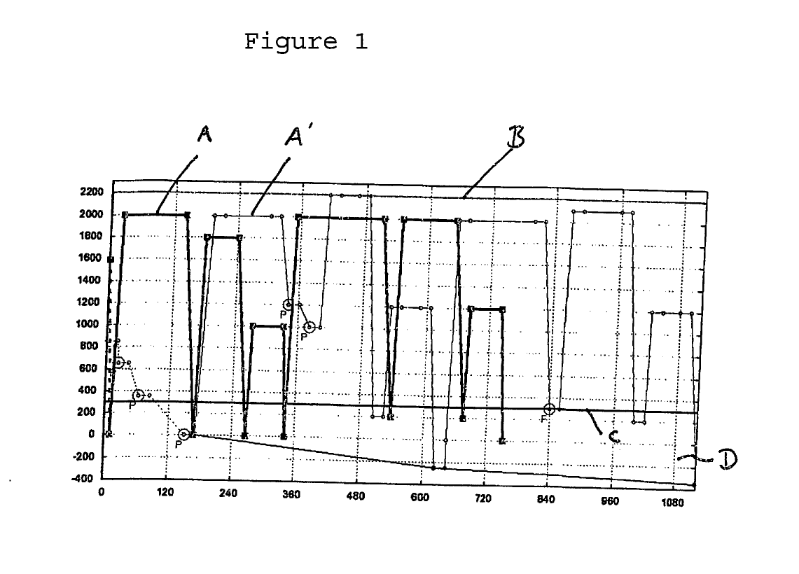

### DETAILED DESCRIPTION OF THE INVENTION

According to the example described below a default prescription with the following parameters is given:
Performing two cycles;
Pressure-controlled initial outflow;
Dwell time of 60 min each;
Inflow of 1000 ml each;
Last inflow of 1250 ml;
Inflow rate of 250 ml/min;
Outflow rate of 200 ml/min.

The determination of the ideal treatment time, i.e. the treatment time, where neither process-related nor patient-related or other delays will occur is as follows:

The initial ideal outflow duration is supposed to be 5 min:

Δt_{outflow,initial,ideal}=5 min

This value may be adjustable or may be preset by the apparatus.

The ideal inflow duration results from the quotient of the volume to be applied to the patient having the preset inflow rate or having the adjusted inflow rate, respectively:

M_{inflow, ideal}=V_{inflow}/Q_{inflow}=1000 ml/250 ml/min=4 min.

The ideal outflow duration results from the quotient of the dialysis solution volume to be withdrawn with the preset or the adjusted outflow rate. If one considers the volume to be withdrawn is equal to the volume to be applied, the following results:

Δt_{outflow, ideal}=V_{outflow}/Q_{outflow}=1000 ml/200 ml/min=5 min.

One cycle is composed of an inflow, a dwell period as well as an outflow. The ideal cycle duration Δt_{cycle, ideal} is:

Δt_{cycle, ideal}=Δt_{inflow, ideal}+Δt_{dwell time, ideal} +At_{outflow}, ideal=4 min+60 min+5 min=69 min.

The ideal duration of the last inflow results from the quotient of the inflow volume (1250 ml) and the inflow rate (250 ml/min):

t_{inflow, final, ideal}=V_{inflow}/Q_{inflow}=1250 ml/250 ml/min=5 min.

From this the overall treatment duration Δt_{Gesamt, ideal} results as follows:

Δt_{overall, ideal}=Δt_{outflow, initial, ideal}+2*Δt_{cycle, ideal}+Δt_{inflow, final, ideal}=5 min+2*69 min+5 min=148 min.

Consequently, the ideal overall treatment duration for this example is 148 min. This treatment duration results if there are no process-related delays, such as breaks or delays during inflow or outflow, and no non-process-related delays, such as patient-related delays or delays related to the equipment, which was used.

If process-related as well as patient-related delays now are taken, then an estimated overall treatment duration results for the same course of treatment (2 cycles) as follows:

Δt_{overall, estimated}=Δt_{outflow, initial}+2*Δt_{cycle}+Δt_{inflow, final}

For the duration of the initial outflow, in turn a time span of 5 min is assumed.

The cycle duration Δt_{cycle} in turn results from Δt_{inflow}+Δt_{dwell time+Δt}_{outflow}.

For the inflow a process-related delay Δt_{inflow, delay} of 120 s is assumed. For the inflow, due to reduced catheter performance relating the inflow, a delay by the factor of 110% is furthermore assumed, i.e. the inflow duration, due to a feature of the catheters, is by 10% higher than in the ideal state, where this feature is not present. If on identifies the catheter parameter F_{Δtinflow} relating the delay during inflow, then for the inflow duration the following results:

Δt_{inflow}=V_{inflow}/Q_{inflow}*F_{Δtinflow}Δt_{inflow, delay}=1000 ml/250 ml/min*110/100+120 s=264 s+120 s=384 s=6 min 24 s.

The value for F_{Δtinflow} may be preset to 110% by the apparatus. Basically, it is however also to be taken into account that the value may be set by a user.

For the outflow duration Δt_{outflow} upon delay due to the catheter during outflow F_{Δtoutflow} and upon process-related delay Δt_{outflow, delay} of 60 s the following results:

Δt_{outflow}=V_{outflow}/Q_{outflow}*F_{Δtoutflow}Δt_{outflow, delay}=1000 ml/200 ml/min*150/100+60 s=510 s=8 min30 s.

If for the duration of the last inflow a value F_{Δtinflow} amounting to 110% and a delay of 120 s is assumed again, then the following results

Δt_{inflow, final}=V_{inflow}/Q_{inflow}F_{Δtinflow}=1250 ml/250 ml/min*110/100+120 s=450 s=7 min.

The values Δt._{inflow, delay} and Δt_{outflow, delay} may be due to process-related delays, such as for example recording the patient's pressure, changing the bag during inflow etc.

The values F_{Δtinflow} and F_{Δtoutflow} may for example vary between a minimal value (100%) and a maximal value (200%). For both values a default value, for example 130 % may be set or suggested, respectively.

Consequently, the cycle duration for an assumed dwell time of 60 min now is as follows:

Δt_{cycle}=384 s+3600 s+510 s=4494 s=74 min 54 s.

For two cycles the overall treatment duration is obtained according to:

Δt_{overall, estimated}=Δt_{outflow,initial}+2*Δt_{cycle}+Δt_{inflow, final}=300 s+2*4494 s+450 s=9708s=161 min 48 s≈162 min.

If one considers for the value of F_{Δtoutflow} to be editable, i.e. to be user-changeable, the following table results for the outflow duration and for the estimated overall duration of the treatment for different values for F_{Δtoutflow}:

| F_{Δtoutflow} [%] | Δt_{inflow} [s] | Δt_{overall, estimated} [min] |
|---|---|---|
| 100 | 360 | 157 |
| 110 | 390 | 158 |
| 120 | 420 | 159 |
| 130 | 450 | 160 |
| 140 | 480 | 161 |
| 150 | 510 | 162 |
| 160 | 540 | 163 |
| 170 | 570 | 164 |
| 180 | 600 | 165 |
| 190 | 630 | 166 |
| 200 | 660 | 167 |

If it is furthermore considered for time spans for one or more breaks Δt _{break} and/or messages Δt_{message} to be taken into account, then the estimated cycle duration as well as the estimated overall duration is extended accordingly. The estimated duration of the break may thereby match to the specified duration of the break. The same is true for the dwell time of the dialysis solution in the patient. For the duration of a message the value of zero may be assumed.

As stated above, the initial outflow volume V_{outflow,initial} may be estimated for a pressure controlled initial outflow. The estimation may be done with the help of the following relation:

V_{outflow,initial}=Δt_{outflow,initial, eff}*Q_{outflow,initial, eff}

The effective outflow rate Q_{outflow,initial,eff} results from the specified outflow rate Q_{outflow,initial,specified} according to the following:

Q_{outflow,initial, eff}=Q_{outflow, initial, specified}/F_{Δtoutflow}.

The effective outflow duration Δt_{outflow, initial, eff} results from the following:

Δt_{outflow, initial, eff}=Δt_{outflow, initial, specified}+Δt_{outflow, delay}.

For F_{Δtoutflow} and for Δt _{outflow,initial, specified} for example the values of 110% and 5 min may be employed.

According to this relation the initial outflow volume V_{outflow,initial} may be determined.

According to course A the figure shows the course estimated by the process according to the invention; and the course A′ shows the actual course of the volume in the patient of the dialysis solution over the time. The referential marks P symbolize print events.

As it may be seen from the figure the course A indicated as a dotted line starts from the determined initial outflow volume and extends over a defined period of 5 min.

Following an inflow phase the in-patient dwell time starts, followed by an outflow phase to zero the volume. As it may be seen from the figure, inflow phases, dwell phases and outflow phases will subsequently be repeated.

The line B characterizes the maximum allowable volume of the dialysis solution in the patient, the line C characterizes the maximum allowable volume remaining in the patient.

Area D characterizes the ultrafiltration volume withdrawn during the treatment. The initial outflow phase will not be taken into account in determining of the ultrafiltration volume.

A tendency analysis over extended periods of time could predict if and how the characteristics of the catheters change over the time. The most effective flow rates of outflow and inflow correspond to the slopes of the course of A′. If the slope is zero then neither outflow nor inflow will occur. This is for example the case in the dwell phases or treatment breaks or process-related down times.

Preferably, each treatment will be documented. The documented report may comprise the average effective flow rate for the inflow and outflow, i.e. the average effective inflow and outflow rates, which are determined for the overall treatment.

The determined values only account for the flow rates where a volume has effectively been transferred in and out of the body, respectively, i.e. the times, where the pumps operate. The effective flow rates may furthermore be documented for all phases. As stated above, from comparing different effective flow rates at different times the catheter performance may be inferred.

1. A process for the estimation of treatment duration of peritoneal dialysis treatment, characterized in that the estimation of treatment duration is performed based on ideal treatment duration, wherein the ideal treatment duration is increased by one or more delay values, wherein the delay values depend on one or more apparatus-specific and/or patient-specific and/or process-specific parameters.

2. The process according to claim 1, characterized in that the ideal treatment duration, i.e. the ideal duration of the initial outflow, comprises the ideal cycle duration multiplied by the number of cycles as well as the ideal duration of the last inflow.

3. The process according to claim 1, characterized in that a patient-specific parameter is formed by the catheter performance.

4. The process according to claim 3, characterized in that the catheter performance is taken into account during estimation of the inflow duration and/or during estimation of the outflow duration and/or during estimation of the duration of the last inflow, wherein preferably provision is made for the catheter performance to be taken into account in the form of absolute or relative increase of the ideal inflow duration and/or the ideal outflow duration and/or the ideal duration of the last inflow.

5. The process according to claim 4, characterized in that catheter performance and especially absolute or relative increase may be set by a user.

6. The process according to claim 1, characterized in that the initial outflow pressure is performed in a pressure-controlled manner and in that the duration of the initial outflow is preset by the apparatus, or in that the initial outflow is performed in a volume-controlled manner and in that the duration of the initial outflow is determined from initial outflow volume and an outflow rate, wherein preferably provision is made for the value determined in this manner to be increased in order to account for the catheter performance.

7. The process according to claim 1, characterized in that the process-specific parameter is an absolute or relative delay during inflow and/or during outflow.

8. The process according to claim 1, characterized in that the volume of the initial outflow is determined from the effective duration of the initial outflow and from the effective outflow rate.

9. The process according to claim 8, characterized in that the effective duration is a preset duration in addition to a time delay and/or in that the effective outflow rate is determined from the preset outflow rate and a value accounting for the catheter performance.

10. The process according to claim 1, characterized in that for the estimated duration of in-patient dwell time of the solution and/or for the estimated duration of treatment break the estimated duration is used and/or in that for the estimated duration of a message the value of zero is assumed.

11. The process according to one of claim 1, characterized in that the estimated treatment duration is composed of the following estimated time(s): initial outflow duration, inflow duration, outflow duration, in-patient dwell time, duration of treatment break, duration of messages, finale inflow duration.

12. The process according to claim 1, characterized in that the effective inflow rates and/or outflow rates are determined and in that for establishing a tendency analysis by documentation of these data over the time a change in catheter performance is determined, wherein preferably provision is made for estimation of treatment duration of future treatments to be based on the catheter performance which has been changed.

13. The process according to claim 1, characterized in that the dwell time of the treatment is changed depending on the estimated treatment duration or in that it will remain unchanged.

14. The process according to claim 1, characterized in that the estimated treatment duration is compared to an upper and/or lower threshold value and that an indication is given to the user if the estimated treatment duration exceeds the upper threshold value or falls below the lower threshold value.

15. A dialysis apparatus for performing peritoneal dialysis having at least one logic or control unit, at least one input unit to input one or more values, as well as at least one processor, characterized in that the processor is programmed to perform a process according to claim 1.
